(19) United States
(12) Patent Application Publication
(10) Pub. No.: US 2004/0105894 A1
(43) Pub. Date: Jun. 3, 2004

(54) Trace Metals synergized copper nucleotides and copper glycosides for anti-aging and antiviral compositions

(76) Inventor: Shyam K. Gupta, Scottsdale, AZ (US)

Correspondence Address: SHYAM K. GUPTA, BIODERM RESEARCH, 5221 E. WINDROSE DRIVE, SCOTTSDALE, AZ, 85254, US

(21) Appl. No.: 10/306,948

(22) Filed: Nov. 29, 2002

(51) Int. Cl. A61K 48/00; A61K 38/16; A61K 38/40; A61K 31/715; A61K 33/24

(52) U.S. Cl. 424/617; 514/6; 514/44; 514/54

(57) ABSTRACT

I have discovered that trace metals such as copper, zinc, iron, and manganese that are necessary for the proper functioning of superoxide dismutase (SOD) and other deactivators of active-oxygen molecules (which cause aging of skin and other skin disorders), can be delivered from the topical compositions. This is achieved by the preparation of copper and other trace metal complexes with phosphorylated nucleosides, such as nucleotides, and phosphorylated monosaccharides, such as phosphorylated glycosides which act as small molecular weight (SMW) transporter molecules. These trace metal complexes of nucleotides and glycosides can be prepared by an in-situ method in water, water-miscible organic solvent, or a mixture of water and water-miscible organic solvent from commonly available ingredients in concentrations that are desirable and can be accurately controlled. I have additionally discovered compositions to achieve the transport of copper from the surface layers of skin into the deeper layers of skin utilizing SMW transporter molecules; and the intra-cellular storage of copper ions in the cell, for example in a bound form with glutathione; and the intra-cellular transport of copper from glutathione to SOD apoprotein by metallochaperones; and the supply of energetic molecules, such as ATP, ADP, or phosphorylated saccharides for SOD metallochaperones to perform their intra-cellular metal transfer function. These cosmetic or pharmaceutical compositions are useful for antiaging and antiviral benefits.

### CROSS-REFERENCE TO RELATED APPLICATIONS

Not Applicable.

### STATEMENT REGARDING FEDERALLY SPONSORED RESEARCH OR DEVELOPMENT:

Not Applicable.

### REFERENCE TO SEQUENCE LISTING, A TABLE, OR A COMPUTER PROGRAM LISTING COMPACT DISK APPENDIX:

Not Applicable.

### BACKGROUND

Maintaining a youthful appearance is of great importance to many people, particularly in an aging population. Several of the visible signs of aging result from its effects on the skin. The passage of time is reflected in the appearance of wrinkles and fine lines; by a slackening of tissue; a loss of cutaneous elasticity; a leathery or dry appearance; by the yellowing of the skin which becomes duller and loses its radiance; and the appearance of age-spots that are especially visible in face, neck, chest, and arms. Skin that has been consistently exposed to sunlight throughout life may show pigmentation marks, telangiectasia and elastosis. At the histological level, skin damage from photo aging is shown in tangled, thickened, abnormal elastic fibers, decreased collagen and increased glycosaminoglycan content. The aging process also results in thinning and deterioration of the skin, and hair loss. There is a reduction in cells and in blood supply, and a flattening in the junction between the dermis and epidermis.

Treatments designed to prolong or promote youthful appearance include topical applications of cosmetic preparations, lotions and moisturizer, electrical stimulation, collagen injections and cosmetic surgery. However, there is still a serious need for skin care compositions that treat wrinkles and fine lines, and restore the youthful appearance of the skin. A number of novel approaches are already known, for example U.S. Pat. No. 6,436,416, to Grainger; U.S. Pat. No. 6,328,987, to Marini; U.S. Pat. No. 5,538,945, to Pallenberg; U.S. Pat. Nos. 5,888,522 and 5,164,367, both to Pickart; U.S. Pat. No. 6,444,647, to Robinson, U.S. Application 20020136763, to Demopoulos; and U.S. Application 20020102285, to Bishop.

Most of prior art methods to treat aged skin have been based on purely organic compounds. The role of bioinorganic and bio-organic metal molecules in the treatment of skin disorders related to the biological processes of aging is now being understood in greater detail, and recognized by the scientific community. In recent years it has become clear that transition metals, especially copper, are essential for normal development and function of human cells. Copper is the third most abundant trace element in human body, with vitamin-like impact on living systems. Copper functions as a cofactor in over 30 enzymes. The ability of copper to cycle between oxidized Cu²⁺ and reduced Cu⁺ states is used by cuproenzymes involved in redox reactions, the two most important examples being Cu/Zn superoxide dismutase and cytochrome C oxidase. As will become clearer in the later sections of present invention, Cu/Zn superoxide dismutase is an important enzyme responsible for the destruction of toxic superoxide anion in human body that directly relates to the processes of skin aging. The enhancement or increment of SOD functions for antiaging and anticancer benefits is of current scientific and consumer interest. Some of these aspects have recently been disclosed by several authors in recently published text books, such as Valentine et al. [(Advances in Protein Chemistry, vol. 60, pp. 93-121, Academic Press, CA (2002)]; and Massaro [(Handbook of Copper Pharmacology and Toxicology, Humana Press, NJ (2002)], which are quoted here only for reference. It has also become clear that ATP, a major nucleotide present in human body, plays a major role in copper transport, in the form of copper transporting ATPase enzyme, that utilizes the energy of ATP-hydrolysis to transport copper from the cytosol through various cell membranes [Tsivkovskii et al. (J. Biol. Chem., 277, 976-983 (2002); Nakayama et al. (Oncology Reports, 8, 1285-1287 (2001); Wunderli-Ye et al. (Biochem. Biophys. Res. Commun., 280, 713-719 (2001)] These disclosures point to possible importance of nucleotide complexes of copper in the bioavailability and cellular transport of copper in humans. Wijnhoven, et al. (U.S. Pat. No. 6,277,605) disclose an interesting role of divalent metals, such as copper, zinc, and manganese, in the complexation with DNA molecules that results in the bond distance increase of nucleic acid components, resulting in the annealing of the DNA helix. A simple oxidation-reduction step of such divalent metal ions can cause annealing or reannealing of such separated DNA strands. This indicates a prospective application of copper zinc, and manganese complexes of nucleic acids, nucleosides, and nucleotides in cosmetic and biomedical control of the process of skin aging.

It is thus not surprising that there has been much interest in bioinorganic chemistry of copper. A number of copper-based skin care ingredients and pharmaceuticals have been developed by a number of researchers worldwide. Since it is the object of present invention to disclose certain novel applications of copper based bioinorganic ingredients, it is worthwhile to briefly describe various bioinorganic states in which copper can be found in biological systems. It is of further importance, since such various forms of copper can have significantly different biological or cosmetic functions. Copper biomolecules can occur in four types of copper centers. These four copper types, and their characterization methodologies, are identified in Table 1.

TABLE 1
Types of Copper Sites in Biomolecules

| Copper Type | Main Characteristics |
|---|---|
| Copper (I) | Colorless, diamagnetic, epr silent |
| Normal Copper (II) | Visible and epr spectra typical of tetragonally |
| | Coordinated Cu²⁺ |
| Blue Copper (II) | The epr shows abnormally small A₁₁; very |
| | intense absorption (ε about 5000) at 600 nm. |
| Coupled (Cu^{II})₂ | Abnormal visible spectrum; |
| | Diamagnetic and epr silent |

While many copper biomolecules contain copper in only one form, for example “blue” or “normal”, there are also numerous cases where several different types of copper are present and that can provide difficulties in working out their mode of action, or even their applications. From the data in Table 1, it is clear that the identification of specific copper species, when several different types of such species may be present, is not an easy task. Yet such species may have a different biological role. This is mentioned here because it is another object of present invention to prepare copper biomolecules that are distinct in their chemical state.

The “normal” copper (II) sites are those in which Cu²⁺ ion is coordinated by a square set of ligands, usually all nitrogen atoms, such as those present in imidazole moiety of one (or several) histidine molecules. There may be additional ligands occupying more distant coordination sites above and below that square plane of nitrogen ligands. Such copper (II) sites are easily identified by spectral analysis of such copper complexes. The active site of bovine Superoxide Dismutase enzyme, one of the best-known examples of “normal” copper (II) site, is illustrated in FIG. 1. All bond distances are in Angstrom units. It is to be noted that this active site also contains zinc as a cofactor. It is to be noted that copper in such “normal” copper (II) sites is electronically bound to four different nitrogen atoms. (FIG. 1. Chemical Structure of Active Site of Superoxide Dismutase Enzyme.)

The “blue” copper (II) state entails environment quite unlike those in “normal” copper (II) tetragonal complexes. Numerous sophisticated spectroscopic analyses have been made of both the biomolecules themselves and their model systems. However, only X-ray crystallographic data are most reliable. The active site of a “blue” copper (II) biomolecules is shown in FIG. 2. All bond lengths are shown in Angstrom units. It is to be noted that copper in “blue” copper (II) sites is electronically bound to four different atoms, two of which are nitrogen and two of which are sulfur atoms. (FIG. 2. Chemical Structure of the “Blue” Copper (II) Active Site.)

Coupled (Cu^{II})₂ is found most commonly in respiratory proteins of phyla Mollusca and Anthropoda, for example squid, octopus, lobster, and crabs. These proteins, called hemocyanins, are very large that contains subunits. Each subunit contains a pair of Cu atoms, and those atoms can bind one molecule of oxygen per pair of copper atoms. The two-copper active site of hemocyanins is also found in enzyme tyrosinase. In humans this enzyme converts phenols to catechols that leads to the eventual formation of skin pigment, melanin. It is to be noted that copper in “coupled” (Cu^{II})₂ is electronically bound to a minimum of four different atoms, two of which are nitrogen and two of which can be oxygen (see FIG. 3). (FIG. 3. Oxygen Coordination of Coupled Cu in Tyrosinase Enzyme.)

From the discussion above and the inspection of FIGS. 1, 2, and 3, the following points are clear so far: these points shall become clearer in the Objects of the Invention section of this disclosure;

(i) Antiaging enzyme superoxide dismutase contains copper (II) in its active site;

(ii) Copper in copper enzymes can be found in several distinctly different chemical states, each of which has a specific function;

(iii) Copper in excessive amounts in a cell, present in a free state, can cause cellular toxicity;

(iv) Copper generally requires four coordination sites in metalloenzymes, all four of which can be nitrogen, or two of which can be nitrogen and the other two can be sulfur or oxygen atoms from appropriate donor ligands;

(v) Superoxide dismutase also requires zinc as a cofactor;

(vi) An energy donor, such as Adenosine Triphosphate (ATP) is required for the transfer of copper from cytosol to superoxide dismutase enzyme;

(vii) It is clear to see that copper (II) can also bind with sulfur ligands, in addition to nitrogen atoms; and

(viii) From the example in FIG. 3 for tyrosinase enzyme, it is clear to that copper (II) can also bind with oxygen ligands, in addition to nitrogen atoms.

Of over 30 enzymes that require copper in their active-site, superoxide dismutase is most important from the viewpoint of skin aging and inflammation. Superoxide dismutase (SOD) is one of the enzymes that are most directly linked to superoxide anion detoxification, and, as its production slows down, the process of aging accelerates. Among other biologically important cuproenzymes, the formation of elastin and collagen is a function of amine oxidase, which is another example of a copper-containing enzyme. The skin pigmentation, or melanin formation, is a function of tyrosinase, which is a copper-based monooxygenase class of enzyme. Ceruloplasmin, a copper-containing metalloenzyme, has a role in the iron transport in human body. Dopamine hydroxylase, another copper-based enzyme, is present in adrenal glands, and it converts dopamine to norepinephrine. SOD occurs in three distinct forms in mammalian systems;

(i) SOD containing copper and zinc (CuZnSOD, SOD1), which is usually located in the cytosol;

(ii) SOD containing manganese (MnSOD, SOD2), which is usually located in mitochondria (MnSOD); and

(iii) Another SOD containing Cu and Zn (CuZnSOD, SOD3), which is found in extra-cellular spaces.

(iv) Additionally, many bacterial SOD contain iron.

In mammalian systems, CuZnSOD (SODI) catalyses the dismutation of the superoxide anion radical (O₂^{-•}) according to Equations 1 and 2;

O₂⁻+Cu(II)ZnSOD→O₂+Cu(I)ZnSOD (Equation 1)

O₂⁻+Cu(I)ZnSOD+2H⁺→H₂O₂+Cu(II)ZnSOD (Equation 2)

One product of this reaction, H₂O₂, is also a harmful substance. Hydrogen peroxide is removed by the heme iron metalloenzymes catalase according to Equation 3;

2H₂O₂→2H₂O+O₂ (Equation 3)

The superoxide anion (O2⁻•) exhibits numerous physiological toxic effects including endotelial cell damage, increased microvascular permeability, formation of chemotactic factors such as leukotrienes, recruitment of neurophils at the sites of inflammation, lipid peroxidation, and oxidation, release of cytokines, DNA single-strand damage, and formation of peroxynitrite anion (ONO₂^{-•}^{₋}), a potent cytotoxic and pro-inflammatory molecule generated according to Equation 4;

O₂^{-•}+NO→ONO₂^{-•} (Equation 4)

Excess superoxide anion can also lead to the formation of highly oxidizing species such as hydroxide and peroxide radicals. Superoxide radical anion, and the peroxynitrite anion formed in its reaction with NO, cause cell death from ischemic tissue. Most of these physiological effects lead to skin aging and tissue degeneration (Macarthur et al., Proc. Natl. Acad. Sci. USA, 97, 9753-9758 (2000). In this capacity, SOD acts as an antioxidant inhibiting aging and carcinogenesis.

Preventing tissue and cell damage caused by reactive oxygen species in mammals has received wide scientific interest, as stated by Hellstrand et al. (U.S. Pat. No. 6,462,067. Free radicals such as superoxide ions, hydroxy radicals, oxides are known as a major factor of degeneration and thus the ageing of the skin. They destruct the proteins and lipids of the cellular membrane, affect the DNA and also decompose the hyaluronic acid, a key substance of the skin. Under normal biological conditions there is an equilibrium ratio between the free radicals coming up and their embankment by endogenous chemical or enzymatic systems. Additional outside stress factors such as aggressive atmosphere, tobacco smoke, ultraviolet radiation etc. may overload these inherent immune systems and shift the equilibrium in favor of the free radicals. Inflammation or ageing phenomena of the skin may occur, indicating a need for compensation by cosmetic products. Among principal enzymes that have an effect on aging process, catalase, glutathione peroxidase, ascorbate peroxidase, superoxide dismutase, glutathione peroxidase, and ascorbate peroxidase are most important. The promotion of superoxide dismutase as a method to control various human ailments including aging has been studied extensively, for example Dugas et al. (U.S. Pat. No. 6,426,068), Anggard et al. (U.S. Pat. No. 6,455,542), Hellstrand et al (U.S. Pat. Nos. 6,462,067 and 6,407,133), Golz-Berner et al. (U.S. Pat. No. 6,426,080), and others. Medical researchers have attempted to design low-molecular weight SOD mimics (synzymes) that would mimic the natural SOD enzyme in removing superoxide anion, O₂⁻•, and the perhydroxyl radical, HO₂., as well as preventing formation of peroxynitrite anion, ONO₂⁻•.

It is well recognized that metalloenzymes and protein-based metal complexes are too large in their molecular weight to be useful for any topical applications where high bioavailability is desired. Such molecules have thus found applications in areas such as wound healing where their presence on skin surface is more beneficial, and their absorption into deeper layers of skin is not desired. It is for this reason that such molecules have not found applications in areas that require their enhanced bioavailability into deeper layers of skin, for example anti-aging, collagen synthesis enhancement, and skin whitening. Superoxide dismutase itself has been used in topical applications for antiaging products. However, the molecular weight of this enzyme is so large that its penetration into deeper layers of skin is highly unlikely. Any perceived benefits are most likely the inadvertent result of the separation of copper from the enzyme itself and its subsequent absorption into the skin. This separation of copper from superoxide dismutase in topical products can result from various chelating agents that are used in such compositions.

In order to circumvent the difficulties encountered in the bioavailability of metalloenzymes and protein-based metal complexes from topical applications, including complexes that contain copper or zinc, smaller molecular weight models that mimic the active site of larger molecular weight metalloenzymes have been extensively studied and reported by, for example, Pickart et al. (U.S. Pat. Nos. 5,858,993; 5,888,522; 5,698,184; 5,550,183; 5,554,375; 5,164,367; 4,665,054; 4,760,051; 4,810,693 and 4,877,770); Pallenberg et al., (U.S. Pat. Nos. 6,017,8880 and 5,538,945); and Lawyer et al., (U.S. Pat. No. 6,042,848). Other biomimetic superoxide dismutase models include complexes in which copper has been replaced with an isosteric manganese atom. The preparation of these biomimetic models is very difficult, and many such compositions are not suitable for cosmetic applications. Moreover, it is to be noted that despite the therapeutic promise of the above-mentioned metal complexes, toxicity and tissue irritation occur with many metal complexes. For example, while copper-salicylate complexes and numerous copper-salicylate analogs possess anti-inflammatory activities, other salicylate analogs such as the copper (II) complex of salicylaldehyde benzoyl hydrazone are highly toxic to tissues. Similarly, copper(II)-Gly-L-His-L-Lys supports cellular viability and possesses anti-inflammatory and healing actions, yet close synthetic aroylhydrazone analogs of its copper-binding region are extremely toxic to cells and tissues.

Despite extensive efforts in developing smaller molecular weight models of SOD enzyme, especially those mentioned above, none have proven fully efficient or effective. This is due to the fact that these prior art disclosures have focused only on the aspect of copper bioavailability. For example, the smaller molecular weight models, such as copper peptides and copper amino acids, provide only the enhanced bioavailability of copper. These disclosures do not provide any additional support to enhance SOD efficacy, such as the inclusion of a component, such as glutathione, for the intracellular storage of copper or other necessary trace metal ion. They also do not provide any provision, such as ATP, ADP, or phosphorylated glycosides, for extra energy that is required for the transport of copper from the storage molecule to the apoprotein of SOD metalloenzyme. These also do not provide the other trace metals, such as zinc or iron that are required as necessary cofactor. These also frequently do not provide molecules that have distinct and established chemical structures. Also, most of these disclosures provide copper transport systems that are deactivated by chelating agents and sequestrants that may be present in a topical composition. These copper derivatives, in most cases also cause significant oxidation of other organic chemicals present in a topical composition, resulting in off-odor formation, product discoloration, and decomposition of certain essential ingredients.

As has become known only very recently since 1999 that there are several additional factors which are responsible for the transport and utilization of copper in biological systems. The cellular transport of copper from ingestion mode has recently been reviewed by Sarkar et al [(Chem. Rev., 99, 2535-2544 (1999)] and summarized in FIG. 4. (FIG. 4. Cellular Transport of Copper via Ingestion Mode.) Copper ions are first bound to metal transporter molecules that carry metal ions across cell membranes. For example, human copper transport protein receives copper(I) ions on the cell surface and transports them into the cell cytosol. In biological terms, copper is absorbed from gastrointestinal tract and enters an inter- and intracellular exchangeable pool. During uptake, copper is reduced to copper (I) and absorbed by the cell via copper transporter, for example, human copper transporter (hCtr). After transport by the Ctr protein, copper ions are stored in biomolecules such as glutathione. Cytoplasmic Cu(I)-glutathione (Cu(I)GSH in FIG. 3) then donates copper to various copper chaperone proteins that deliver copper to metalloenzymes such as superoxide dismutase. It is thus amply clear from these reports that;

(i) Incorporation of a copper storage biomolecule, such as glutathione, is critical for the storage of copper in the cytosol, and its subsequent transport by transport proteins to metalloenzyme, superoxide dismutase. Any copper stored in a cell in a free, unbound state can cause copper toxicity,

(ii) Copper transport proteins, Ctr, are too large in their molecular weight to be of any practical utility in topical applications of copper; and,

(iii) Smaller molecular weigh transporter molecules will be required for the transport of copper from the upper layers of skin into the deeper layers of ski.

The transport of copper from intracellular copper storage molecules such as glutathione or metallothioneins to apoprotein of SOD is performed by protein molecules called metallochaperones. The concept of metallochaperones is of very recent origin. In a recently published book, Roat-Malone [Bioinorganic Chemistry—A Short Course, Wiley-Interscience, NJ (2002)] describes the importance of metallochaperones in the activation of superoxide dismutase. To illustrate this point, it is well known that enzyme superoxide dismutase binds copper with great affinity. This affinity is so great that total free cytoplasmic copper ion concentration is less than 10⁻¹⁸ M, or less than one copper ion per living cell. In kinetic terms, less than 0.01% of the total cellular copper becomes free in the cytoplasm during the lifetime of the cell. Despite high cellular capacity for copper uptake and chelation, metallochaperones succeed in acquiring copper and delivering it to metalloenzymes that require it. This transportation of copper from the copper storage molecule to SOD apoprotein by metallochaperone requires energy, perhaps from energetic molecules such as ATP or ADP, since copper ATPases act as metallochaperones for SOD. The structure of copper ATPase is of ferredoxin-like large molecular weight complexity, and hence not suitable for any topical delivery systems.

From the above, in summary, the following four types of ingredients are required for the most efficient utilization of copper ions by SOD from any topical delivery system;

(i) the transport of copper from the surface layers of skin into the deeper layers of skin utilizing smaller molecular weight transporter molecules;

(ii) the storage of copper ions in the cell, for example in its bound form with glutathione or a metallothionein;

(iii) the transport of copper from glutathione to SOD apoprotein by metallochaperone; and,

(iv) The supply of energetic molecules, such as ATP or ADP, for SOD metallochaperone to perform their metal transfer function.

Another problem with copper complexes for therapeutic use concerns the binding affinity of copper ion to the complexing molecule. While a defined copper-complex can be synthesized, its therapeutic use places it in the physiological milieu of the tissues where a plethora of literally hundreds of compounds compete for binding to the copper ion, which can form electrostatic bonds to as many as six separate molecules. If the copper is removed from the complex and becomes loosely bound, then tissue irritation occurs. Further complications arise when such metal complexes are formulated into carrier creams or ointments. Various chemicals are added to the formulations to increase adherence to skin and wound surfaces and to enhance the penetration of the complexes into the target tissue. Yet, since many of these substances, for example chelating agents, also bind to the metals, the expected therapeutic benefits may be nullified or significantly attenuated. Thus, the composition of copper nucleotides should be such that they are not deactivated by other common ingredients present in topical formulations, such as chelating agents, sequestrants, and such.

A yet another problem exists for the development of any topical delivery systems for copper and other trace metals. It is well known that trace metals such as copper, iron, and manganese can catalyze extensive oxidation of fatty organic ingredients that are commonly present in topical preparations in the presence of air. Such oxidation results in the product discoloration and malodor formation. Additionally, any skin beneficial ingredients that are present in such formulations can also decompose or transform into non-functional materials from such oxidation. It is thus very common to use chelating agents such as EDTA in cosmetic compositions to bind with copper and iron in order to prevent such oxidation. The use of such chelating agents is also known to deactivate a number of previously reported low molecular weight copper transporting ingredients such as copper peptides and copper amino acids. It would thus be highly desirable to develop low molecular weight copper transporting ingredients for topical applications that are not deactivated by the chelating agents, and that do not cause the oxidation of other ingredients in such topical compositions.

### OBJECTS OF THE INVENTION

It is the object of this invention to develop low molecular weight (LMW) transporters of copper and other trace metals necessary for cellular functions, and their utilization in topical anti-aging and antiviral compositions.

It is another object of this invention to develop simple, in-situ preparation of such LMW trace metal transporter molecules from commonly available ingredients.

It is another object of this invention to provide trace metal transporter molecules that contain such trace metals in predetermined and known chemical forms, and in known quantities.

It is another object of this invention to develop LMW trace metal transporter molecules with high bioavailability that are easily absorbed through skin from topical applications and transport such metals into the deeper layers of skin.

It is another object of this invention to develop LMW trace metal transporter molecules that are not affected by other ingredients, such as chelating agents and sequestrants that may be present in the topical compositions for other purposes.

It is another object of this invention to provide LMW trace metal transporter molecules that are stable under ordinary conditions of their manufacture and storage.

It is another object of this invention to include trace metal intra-cellular storage molecules to provide the storage of trace metal ions in the cytosol after such ions have entered the cell in their bioavailable LMW metal transporter form.

It is another object of this invention to include energetic molecules to provide energy for the intra-cellular transport of trace metals from their storage molecules to the apoprotein of metalloenzymes by metallochaperones .

It is another object of this invention to provide additional trace metals that may be required as cofactors (such as zinc, iron, and manganese) that provide synergistic benefits in combination with LMW trace metal transporter molecules in topical compositions.

It is another object of this invention to provide LMW trace metal transporter molecules that are new and not known in the prior art.

### BRIEF DESCRIPTION OF THE INVENTION

I have discovered that trace metal derivatives of phosphorylated nucleosides and sugars, such as nucleotides and phosphorylated mono-saccharides, for example, adenosine triphosphate (ATP), adenosine diphosphate (ADP), adenosine monophosphate (AMP), fructose-1,6-diphosphate, and glucose monophosphate, act as transporters of such metals in topical compositions from the surface layers of skin into the deeper layers of skin. These transporter molecules are new and not known in the prior art as transporter molecules for topical compositions.

I have additionally discovered that it is essential that such nucleotides and glycosides have at least one phosphorylated chemical entity present for binding with the trace metal component. Additional binding or chelating centers, such as nitrogen and sulfur moieties, may also be present. These metal nucleotides have distinct and known chemical composition that is predetermined by the known composition of commonly available ingredients that are used in their preparation.

I have additionally discovered that such trace metal derivatives of nucleotides and glycosides can be prepared from readily available ingredients by an in-situ method without requiring any special equipment or expensive technology.

I have additionally discovered that such compositions can be formulated with a metal storage molecule, such as glutathione. Glutathione, in such applications, can additionally contain a cofactor metal ligand, if so desired, for any synergistic benefits.

I have additionally discovered that ATP, ADP, AMP, and phosphorylated mono-saccharides also act as energetic molecules after their entry into the cytosol along with trace metal that are bound to them. This is in addition to their function as transporter molecules for such trace metals.

I have additionally discovered that trace metal nucleotides and glycosides of present invention are stable in cosmetic compositions, even in the presence of chelating agents and sequestrants, and they do not cause any excessive oxidation or decomposition of other constituents as may be present in such topical compositions.

### BRIEF DESCRIPTION OF THE DRAWINGS

The following four drawings that represent FIG. 1, FIG. 2, FIG. 3, and FIG. 4 are attached.

FIG. 1. Chemical Structure of Active Site of Superoxide Dismutase Enzyme.

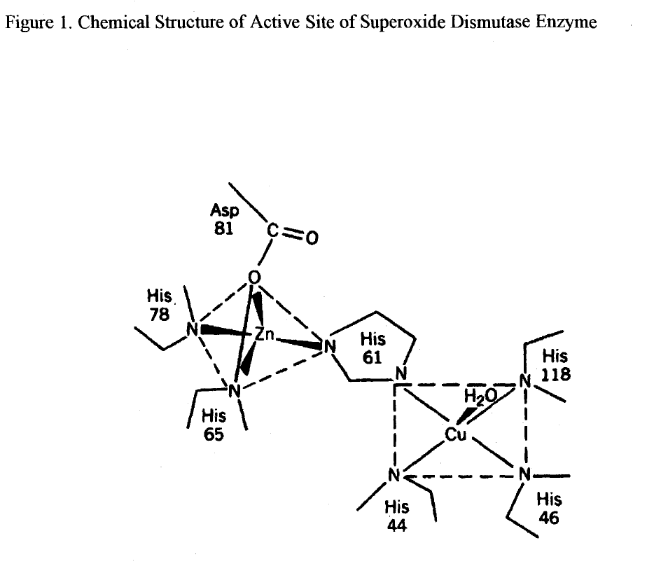

FIG. 2. Chemical Structure of the “Blue” Copper (II) Active Site.

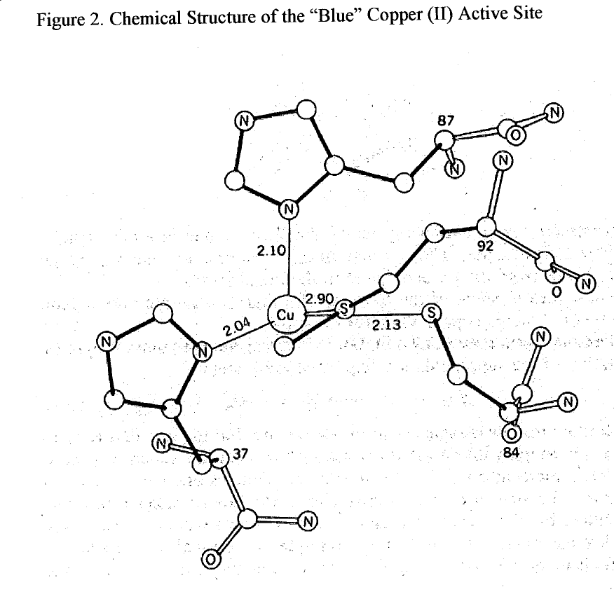

FIG. 3. Oxygen Coordination of Coupled Cu in Tyrosinase Enzyme.

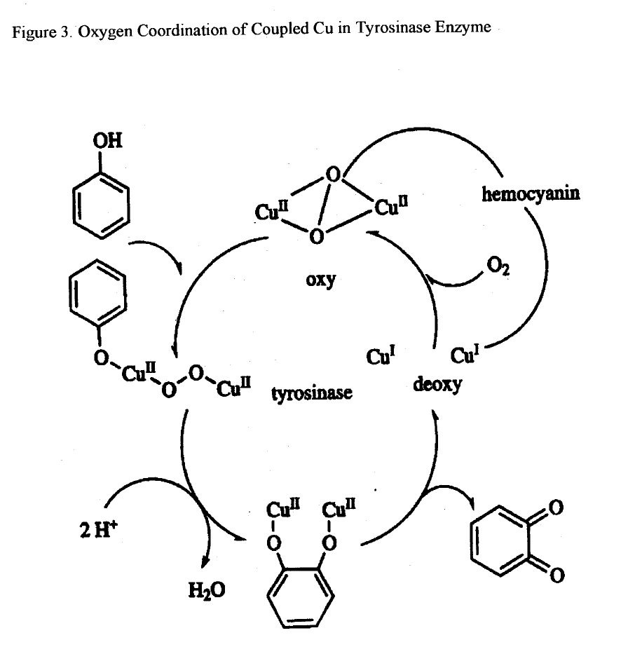

FIG. 4. Cellular Transport Mechanism for Copper via Ingestion Mode.

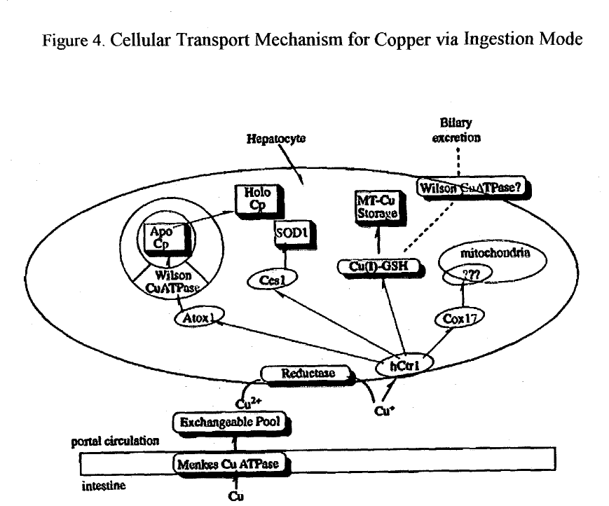

### DETAILED DESCRIPTION OF THE INVENTION

Superoxide dismutase (SOD) is one of the most important metalloenzyme that is linked with the control of the process of aging and carcinogenesis in man. This metalloenzyme contains both copper and zinc at its active site. The following key moieties are required for its proper functioning in a cell;

(i) A source of copper;

(ii) A transporter(s) of copper from extra-cellular to intra-cellular levels;

(iii) A storage device for copper within the cell;

(iv) A chaperone to transport copper from the storage molecule to the apoprotein of SOD enzyme;

(v) An energy source for the transport of copper from copper storage molecule to the apoprotein of SOD (which, in many cases, is copper ATPase); and,

(vi) Additional cofactor trace metals, such as zinc, manganese, and iron.

While the transport of copper from digestive system can be performed by transport proteins, the transport of copper and other trace metals from skin surface via topical delivery systems with transport proteins is not practical, as such large molecular weight copper carrier proteins can not absorb and penetrate through the upper layers of skin. Smaller molecular weight transporter molecules must thus be devised for topical systems to transport trace metals from the upper layers of skin into the deeper layers of skin. Similarly, the delivery of chaperone proteins from topical preparations is at present not technologically feasible because of their very large molecular weight.

I have discovered that trace metals such as copper, zinc, iron, and manganese that are necessary for the proper functioning of SOD and other deactivators of active-oxygen molecules can be delivered from the topical compositions. This is achieved by the preparation of copper and other trace metal complexes with phosphorylated nucleosides and phosphorylated mono-saccharides, such as nucleotides and glycosides. These trace metal complexes of nucleotides and glycosides can be prepared by an in-situ method in water, water soluble organic solvent, or a mixture of water and water-miscible organic solvent according to the following steps;

(i) Combination of water soluble trace metal donor derivatives that can be inorganic or organic in nature. Using copper as an example, copper chloride, copper sulfate, copper nitrate, copper amino acid chelate, copper EDTA, copper peptide, copper gluconate, copper histidinate, and such can be used. Other derivatives of copper or other trace metals can also be used in this chemical scheme. Such trace metal donor derivatives are combined with a trace metal transporter derivative, such as a nucleotide or a phosphorylated mono-saccharide (glycoside). Copper, or other trace metals, are thus transferred from their inorganic or organic donor derivative to the phosphoric acid center of nucleotide or phosphorylated mono-saccharide. The nitrogen centers of nucleotide and hydroxyl centers of glycoside provide further chelating centers to stabilize such trace metal nucleotides or trace metal glycosides. A few examples are shown in Equations 5 to 9;

Cu-Gluconate+ATP→Cu-ATP+Gluconic Acid (Equation 5)

Cu-Histidinate+ADP→Cu-ADP+Histidine (Equation 6)

Cu-Gluconate+DNA→Cu-DNA+Gluconic Acid (Equation 7)

ZnCl₂+Na₂-Fructose-1,6-diphosphate→Zn (Fructose-1,6-diphosphate)+2 NaCl (Equation 8)

CuCl₂+Na₂-Fructose-1,6-diphosphate→Cu (Fructose-1,6-diphosphate)+2 NaCl (Equation 9)

(ii) The solution of trace metal derivative of nucleotide or glycoside thus formed is then added with mixing to the base of topical composition prepared separately to form the desirable composition,

(iii) The solution of trace element derivative of nucleotide or glycoside thus formed can also be stored under ambient storage conditions for a later use, if so desired, and

(iv) The mixing of trace metal donor derivatives with all other ingredients in a single step mixing method to form the final desired composition is also practical for many compositions.

The exchange of trace metal from a trace metal donor, which can include a chelated form of such trace metal, to a low molecular weight (LMW) transporter molecule is both surprising and unexpected. It is well known in the prior art, and well explained by Pickart et al. in various patents quoted above, that chelating agents do not allow the migration of a metal bound to them to another molecule that is generally not recognized as a chelating agent. All of the LMW transporter compositions claimed in the present invention are not commonly recognized as chelating agents. Although not bound by any theory or hypothesis, the selection of a trace metal donor and the LMW transporter molecule is best achieved when the pH, pK, or pK₁ value of the acid part of the trace metal donor is higher than the pH, pK, or pK₁ of LMW transporter molecule. For example, copper-ATP can be made from ATP and copper gluconate (which is made from gluconic acid and a copper source). The pH of a 1% solution of gluconic acid in water is 2.5. The pH of a 1% solution of ATP in water is 2.0. Therefore, the. pH of gluconic acid solution is higher than the pH of ATP solution. Therefore, when a solution of copper gluconate in water is mixed with a solution of ATP in water, then copper from gluconate moiety migrates to ATP moiety to form Cu-ATP. Cu-ATP is a LMW transporter of copper in the present invention, whereas copper gluconate is not a LMW transporter of copper in the present invention. In another example, a 1% solution of fructose-6-phospate has a pK of 1.2. The glycine moiety in copper glycinate has a pK of 2.34. Therefore, when a water solution of fructose-6-phosphate is mixed with a water solution of copper glycinate, which is a chelated form of copper, then copper migrates from glycine moiety to fructose phosphate moiety to form copper fructose-6-phosphate, a LMW transporter of copper in the present invention. In a yet another example, the pK₁ of ascorbic acid is 4.17, and the pK of glucose-1-phosphate is 1.11. Therefore, by mixing a water solution of zinc ascorbate and a water solution of glucose-1-phosphate, a water solution of zinc glucose-1-phosphate is obtained by the in-situ process of the present invention.

The amount of trace metal that is delivered by the trace metal transported for intracellular functions can vary significantly. This is because various trace metals are required in vastly different amounts for such functions. For example, a human body of approximately 75 kilograms contains only about 250 milligrams, or 3 to 4 parts per million (ppm) of copper ions, whereas there are about 30 to 40 ppm, or about 2 to 3 grams of zinc in the same human body. Because various delivery systems can have a profound effect on how much actual trace metal is delivered in-vivo, it is difficult to exactly calculate how much trace metal is needed in a topical composition. However, this difficulty is highly reduced in the present invention because exact nature and amount of a trace metal in a composition can be determined from the trace metal ingredients that are used in the compositions of present invention. It is thus possible to deliver 3 to 4 ppm of copper or 30 to 40 ppm of zinc in their exact amounts, if so desired, by the present invention by a very simple in-situ preparation method. This has not been possible by prior art disclosures.

The intra-cellular storage molecule for trace metals is generally a sulfur-containing molecule. Glutathione is most useful, although other similar molecules such as N-acetyl cysteine, thioglycolic acid, and metallothionein can also be used. The amount of such storage molecules is in proportion to the trace metal that is being delivered for intra-cellular functions. If the intra-cellular reservoir of storage molecules does not need any supplementation, then no additional storage molecules are necessary in the formulation.

In another surprising discovery of the present invention, both the energy source required for the transport of trace metal from the storage molecule to SOD apoenzyme and the trace metal transporter molecule for the transport of trace metal from skin surface to deeper layers of skin can be the same molecule. For example, ATP, ADP, fructose-1,6-diphosphate, and glucose phosphate can perform this dual function of being the transporters of trace metals through dermal layers and providers of intra-cellular energy source required for the transport of trace metals from their storage molecule to apoenzyme.

Additional ingredients that may be necessary for the formulation of a suitable composition for consumer use can also be included in the compositions disclosed in the present invention. Such ingredients may include rheology modifiers, examples of which include Aristoflex AVC (Ammonium Acryloyldimethyltaurate/VP Copolymer), Structure Plus and Structure XL (Acrylates/Aminoacrylates/c10-30 Alkyl PEG-20 Itaconate Copolymer), Carbomer, Xanthan Gum, Carbopol ETD 2020 (Acrylate C10-30 Alkyl Acrylate Crosspolymer), Rheocin (trihydroxystearin), Hydramol PGDS (PEG-90 Diisostearate), C24-28 Alkyl Dimethicone, and Behenyl alcohol. It may also include skin feel enhancement additives such as various silicones. Examples of silicone derivations, include, without limitation, most organosilicones, organic siloxanes, and their cross polymer (e.g., dimethicone, dimethicone copolyol, cetyl dimethicone copolymer, cetyl dimethicone, stearyl dimethicone, stearoxydimethicone, behenoxydimethicone, alkyl methicone, amodimethicone, dimethicone alkyl betaine, cyclomethicone, polydimethylsiloxane, diphenyldimethyl polysiloxane, silicone elastomers, cyclomethicone and dimethicone crosspolymer, Jeesilc 6056, Dow Corning 2501). Additional skin beneficial ingredients, examples of particular ingredients include oil-soluble skin beneficial ingredients; water-soluble skin beneficial ingredients; hydroquinone, arbutin, hydroquinone derivatives and other skin whitening agents; dimethylaminoethanol (DMEA), alpha-lipoic acid, coenzyme Q10 (ubiquinone), carnosine, and other anti-wrinkle and anti-aging agents; vitamin C; vitamin E; water-soluble vitamin C derivatives, glycolic acid, lactic acid, mandelic acid, and hydroxy acid derivatives; and various sunscreen UVA and UVB blockers such as titanium dioxide, zinc oxide, benzophenone-3, benzophenone-4, ethylhexyl Methoxycinnamate, and such. The amounts of such ingredients are not limited to any specific limitations, as those versed in this art know that such amounts are determined by many factors that include government regulations, consumer preference, cost, marketing targets, efficacy of the composition, and such.

Definitions.

The following terms used in the present invention have the meanings set forth below.

Amino Acid. Any of a group of organic compounds containing the amino group combined with the carboxyl radical.

Apoenzyme. Penultimate form of an enzyme that is not in its active form. A combination of apoenzyme with a cofactor, such as a trace metal, converts apoenzyme into a fully functional enzyme.

Base. A compound that is capable of so uniting with an acid as to neutralize it and form a salt.

Basic. A compound that has base-like properties.

Bioinorganic. A compound of biomedical importance that has an inorganic moiety, such as a metal atom, in its basic structure. The basic structure of this molecule can be organic or inorganic.

Dalton (Da) A Dalton (Da) is a unit of atomic weight, equal to 1/12th the mass of a 12C atom. It is also referred to as an atomic mass unit (AMU). Most common usage is to describe molecular weights of biopolymers in units of kilo-Daltons (KDa). The average molecule weight of an amino acid is approximately 110 Da.

Derivative. A compound formed or regarded as being formed from a specified substance or another compound, usually by partial substitution.

Dialysis. Size of the pores is such that only small molecules (i.e. 3000 Da or less) can pass through them while proteins and other macromolecules cannot.

Dispersion. An emulsion or suspension. Comprise the dispersed substance and the medium it is dispersed in.

Emulsion. Intimate mixture of two incompletely miscible liquids.

Equimolar. Of equivalent molecular weight.

Hydrophilic. Strong affinity for water.

Hydrophobic. Weak affinity for water.

Inorganic. Pertaining to those compounds lacking carbon, but including carbonates and cyanides.

Ligand. A molecule that binds or forms a complex with another molecule. Usually considered to be a small organic molecule (e.g. glucose, ATP, etc.), but can range in characteristics from metal ions (e.g. Ca2+) to a protein (e.g. lysozyme can be considered the ‘ligand’ when it forms a complex with an anti-lysozyme antibody).

Lipophilic. Strong affinity for fats or other lipids.

Low Molecular Weight (LMW). The molecules of size 3000 Da or less that can pass through a dialysis membrane. For the purpose of present invention, the molecule size of LMW is less than 1000 Dalton units.

Miscible. Capable of mixing in any ratio without separation of the two phases. The mixture formed by a miscible liquid or solid is a solution.

Molecular Weight. Total weight of a molecule, usually given in Daltons (Da) or kilo-Daltons (kDa).

Oleophilic. Strong affinity for oils.

Organic. Being, containing, or relating to carbon compounds, especially in which hydrogen is attached to carbon whether derived from living organisms or not.

Organic solvent. A solvent including a carbon compound. Examples include, without limitation, glycerin, PEG-6 (Polyethylene glycol 300), and Methylpropanediol (MP glycol).

Parts Per Million (ppm). The number of parts of a material or molecule in one million parts of a composition. For example, if 1% copper gluconate is added to a composition, then that composition contains 10,000 parts of copper gluconate (or, 1400 ppm of copper ions, since copper gluconate contains 14% copper in it) in one million parts of that composition.

Signs of Skin Aging. These include, but are not limited to, all outward visibly and tactilely perceptible manifestations as well as any other macro or micro effects due to skin aging. Such signs may be induced or caused by intrinsic factors or extrinsic factors, e.g., chronological aging and/or environmental damage. These signs may result from processes which include, but are not limited to, the development of textural discontinuities such as wrinkles and coarse deep wrinkles, skin lines, crevices, bumps, large pores (e.g., associated with adrenal structures such as sweat gland ducts, sebaceous glands, or hair follicles), or unevenness or roughness, loss of skin elasticity (loss and/or inactivation of functional skin elastin), sagging (including loss and/or damage to functional subcutaneous muscle tissue and including puffiness in the eye area and jowls), loss of skin firmness, loss of skin tightness, loss of skin recoil from deformation, discoloration (including under eye circles), blotching, shallowness, hyper pigmented skin regions such as age spots and freckles, keratoses, abnormal differentiation, hyperkeratinization, elastosis, collagen breakdown, and other histological changes in the stratum corneum, dermis, epidermis, the skin vascular system (e.g., telangiectasia or spider vessels), and underlying tissues, especially those proximate to the skin.

Small Molecular Weight (SMW). The molecules of size 3000 Da or less that can pass through a dialysis membrane. For the purpose of present invention, the molecule size of SMW is less than 1000 Da.

Solution. A solid, liquid, or gas mixed homogeneously with a liquid.

Solvent. A substance capable of or used in dissolving or dispersing one or more other substances, especially a liquid component of a solution present in greater amount than the solute.

Suspension. Particles mixed in a fluid or a solid, but undissolved.

Synergism. The joint action of different substances in producing an effect greater than the sum of effects of all the substances acting separately.

Synergistic. Acting together

Trace Metal. Any of certain chemical metallic elements found in very small amounts in plant and animal tissues and having a significant effect upon biochemical processes.

Water miscible organic solvent. An organic solvent that can be mixed with water in any ratio without separation of the water from the organic solvent. In the practice of the invention, the preferred (but not required) water miscible organic solvents are those commonly used in cosmetic applications, for example, glycerin, ethylene glycol, propylene glycol, butylene glycol, hexylene glycol, pyrrolidone, N-methyl pyrrolidone, dimethyl sulfoxide, dimethyl sulfone, polyethylene glycol, polypropylene glycol, methylpropanediol, and similar solvents.

### EXAMPLES.

The following examples are for illustration purposes only, and they do not represent any limitation or scope of the present invention. All compositions are in weight percentages. The color measurements were done on a Hunter Lab color meter. This color meter measures color on a scale defined as L,a,b scale. “L” is a valve from 100 to 0, representing white and black colors (lightness and darkness). L=100 shows indicates white color. L=0 indicates pure black color. A (−) value of “a” indicates green color. A (+) value of “a” indicates red color. A (−) value of “b” indicates blue color. A (+) value of “b” indicates yellow color. Various numeric values of “a”, and “b” indicate the degree of respective colors. The mixed colors are thus indicated by a mixed value of “L,a,b” as will be noted in various examples below. The materials used had the following properties. Adenosine triphosphate disodium hydrate (molecular weight 551 Da), glutathione (molecular weight 307 Da), copper gluconate (molecular weight 453 Da, Cu=14%), copper amino acid chelate (copper 12%), fructose-1,6-diphosphate dicalcium (molecular weight 416 Da), zinc gluconate (molecular weight 455 Da, Zn=14%), manganese gluconate (molecular weight 445 Da, Mn=12%). The analysis of trace metals quoted in ppm in various examples, as noted below, are within +−10%.

### Example 1

### The Preparation of Copper ATP (Cu-ATP) Solution by In-Situ Method

| Ingredient | % |
|---|---|
| Part “A” | |
| 1. Copper Gluconate | 2.25 |
| 2. Deionized Water | 97.75 |
| Part “B” | |
| 1. Adenosine Triphosphate (ATP) Disodium Hydrate | 2.75 |
| 2. Deionized Water | 97.25 |

Procedure: Ingredients 1 and 2 in Part “A” were mixed in a beaker. A clear blue solution was obtained. It had a pH of 4.0, and the color readings were L=36.15, a=−42.07, b=−6.55. These data indicate that “a” had a (−) value (green), and “b” also had a (−) value (blue). This means the solution was greenish blue in color. This was identified as solution, Part “A”. Ingredients 1 and 2 of Part “B” were mixed in a separate beaker. A clear, water-like solution was obtained. It had a pH of 3.1, and the color readings were L=68.32, a=−0.82, b=+0.23. Since both “a” and “b” are negligible numbers (less than 1), that indicates that the sample had no color in it. This was identified as solution Part “B”. Solutions of Part “A” and Part “B” were then mixed. A color change was immediately noted. The solution still remained clear, and no precipitate or discoloration noted. This solution was identified as solution of “Cu-ATP”. This Cu-ATP solution (identified as “C”) had a pH of 3.5, and the color readings were L=53.52, a=−33.58, b=4.19. It had a copper concentration of 1575 parts per million (ppm), or 0.1575%.

Since the Cu-ATP solution “C” obtained above had only half the amount of total copper, compared to solution Part “A”, a fresh solution of copper gluconate was obtained that contained only half the amount of total copper compared to solution Part “A”, but it still had the same amount of total copper as the solution of Cu-ATP obtained above. This fresh solution of copper gluconate was obtained by mixing 1.13 grams of copper gluconate in 98.87 grams of deionized water. The light blue clear solution thus obtained had a pH of 4.1, and the color readings were L=48.26, a=−34.28, b=−7.76. It was identified as solution “D”. A comparison of solution “C” and “D” made above shows that the “b” color reading of solution “C” had become less negative (i.e. “C” had shifted to a lesser blue color, shifting the color to a greenish blue) than that of solution “D. This clearly confirms that copper had coordinated with ATP to form Cu-ATP complex in “C”. Same color change (i.e. turning to a more greenish blue color for sample “C”) was observed visually, as mentioned above. This confirms that the “Lab” color readings were correlatable to visual observations. However, the “Lab” color readings are more quantitative and measurable for exact comparisons. For this reason, the stability of Cu-ATP solution was also measures by this method, as described in Example 2.

### Example 2

### The Stability of Cu-ATP Solution from Example 1

The solution “C” obtained per Example 1 was stored in a beaker with a plastic film wrapped over it. It was stored in full light (fluorescent lamps) under ambient room temperature conditions. The color readings were measured periodically, and any visually observed discolorations, or precipitate formations, if any, were also recorded, as noted below.

| | Initial | 1 Week | 4 Weeks |
|---|---|---|---|
| “L” | 53.52 | 51.35 | 50.54 |
| “a” | −33.58 | −35.38 | −36.08 |
| “b” | −4.19 | −5.16 | −5.56 |

### Example 3

### Preparation of Cu-ATP-Glutathione Complex In-Situ

| Ingredient | % |
|---|---|
| Part “A” | |
| 1. Copper Gluconate | 2.25 |
| 2. Deionized Water | 47.75 |
| Part “B” | |
| 1. Adenosine Triphosphate (ATP) | 2.75 |
| Disodium Hydrate | |
| 2. Deionized Water | 47.25 |
| Part “C” | |
| 1. Glutathione | 1.50 |
| 2. Deionized Water | 48.5 |

Procedure: Mix all “Part A” ingredients. A clear blue solution is obtained. Mix all “Part B” ingredients in a separate container. A clear, water white solution is obtained. Mix all “Part C” ingredients in a separate container. A clear water white solution is obtained. Mix solution of “Part A” with solution of “Part B”. A greenish blue solution is obtained, as in Experiment 1. Add solution of “Part C” to above mixture of solution “Part A” and “Part B”. A bluish green precipitate was immediately formed. The analysis of this precipitate shows that both glutathione and copper to be present. Cu content was 2100 ppm. This shows instant binding of Copper with Glutathione to form the new complex in-situ.

### Example 4

### Calculation of Parts Per Million of Copper in a Composition

First, the parts per million (ppm) of copper content of a copper donor is calculated by;

Cu ppm in Cu Donor (% Cu in Cu Donor×10,000)/100.

Then, Cu donor (%) needed in a composition to meet a required ppm of Cu is calculated by;

% Cu donor needed=(1/Cu ppm in donor)×Cu ppm desired.

For example, a Cu donor, such as Copper amino acid chelate that has a Cu content of 20%, has the following ppm content;

Cu ppm in Cu amino acid=(20×10,000)/100=2000 ppm.

To obtain a 150 ppm level of Cu in a composition, the following % of Cu amino acid chelate will be needed;

% Cu amino acid needed=(1/Cu ppm in Cu amino acid)×ppm desired;

% Cu amino acid needed=(1/2000)×150=0.075%.

The following formula can be used for this calculation;

((63/mol.wt. of Cu source×wt. of Cu source)/total weight of composition)×1000000;

in which, 63 is the atomic weight of copper, “mol. wt. of Cu source” is the molecular weight of copper “donor”, “wt. of Cu source” is the weight of copper “donor” used, “total weight of composition” is the total weight including all other additives, etc. in a composition.

To illustrate, in Example 1, molecular weight of copper gluconate is 453. If 2.25 grams of copper gluconate was used to make a 200 gram composition, identified as “C”. The copper content of “C” is;

((63/453×2.25)/200)×1000000=1564 ppm, or 0.1564%.

### Example 5

### Calculation of % Amount of a Copper Donor Needed for a Specific Parts Per Million Copper Content in a Composition

Use the following formula,

(1/ppm of Cu source)×ppm Cu desired=% Cu source needed

For example, a Cu donor, such as copper amino acid chelate with a Cu content of 20%, has 2000 ppm Cu content, as calculated above. To have 100 ppm of Cu in a lotion or cream product, for example, copper amino acid required is,

(1/2000)×100=0.05%

### Example 6

### Preparation of a Copper Nucleotide Facial Anti-Aging Serum with Zinc and Manganese as Cofactor Trace Metals

| Deionized Water | to 100 |
|---|---|
| Aristoflex AVC | 1.0 |
| Geogard 221 | 0.5 |
| PEG-6 | 20.0 |
| Zinc Gluconate | 0.01 |
| Copper Gluconate | 0.025 |
| Manganese Gluconate | 0.0001 |
| Adenosine Triphosphate (ATP) | 0.2 |
| Glutatbione | 0.1 |
| Fragrance | 0.15 |
| Botanical Extracts blend | 0.25 |
| Silicone Elastomer | 5.0 |

Procedure: All copper donors (copper gluconate, zinc gluconate, and manganese gluconate) were mixed in water to give a greenish blue solution. To this solution, ATP and glutathione were added with mixing. A clear, purplish blue solution was obtained, indicating a color shift and the transfer of copper from its donors to ATP. Aristoflex AVC was then added to it and the mixture mixed for 30 minutes to form a clear greenish blue gel. All other ingredients were then added to it with mixing. A purplish blue gel was obtained. The product had Zn=14 ppm, Cu=35 ppm, and Mn=0.12 ppm.

### Example 7

### The Preparation of a Cu-ATP Anti-Wrinkle Skin Lotion with Zinc and Manganese as Cofactors

| Water | to 100 |
|---|---|
| Mineral Oil | 1.0000 |
| Phenoxyethanol | 0.9000 |
| Glycerin | 3.8000 |
| Deodorized Jojoba Oil | 0.0001 |
| Vitamin E Acetate | 0.0001 |
| Aloe Vera | 0.0001 |
| Panthenol | 0.0001 |
| Methyl Paraben | 0.2000 |
| Propyl Paraben | 0.1000 |
| PGMS-SE | 2.0000 |
| Stearic Acid | 3.0000 |
| Cetyl Alcohol | 1.2000 |
| Caustic Soda | 0.0001 |
| Deionized Water | 1.0 |
| Manganese Gluconate | 0.001 |
| Copper Amino Acid Chelate | 0.025 |
| Zinc Gluconate | 0.01 |
| Adenosine Triphosphate (ATP) | 0.2 |
| Glutathione | 0.1 |
| Fragrance | 0.6 |
| Botanical Extract | 0.65 |

Procedure: All copper donors were dissolved in water to give a clear greenish blue solution. ATP and glutathione were then added to it. The color changed to purplish blue. This solution was then added to “skin lotion base” with mixing, and all remaining ingredients were also added. A sky blue lotion was obtained. Skin lotion base was obtained by mixing all other ingredients together, then heating at 70 to 80C for one hour, then cooling to ambient temperature with mixing. A white lotion was obtained which contained Cu=30 ppm, Zn=14 ppm, and Mn=1.2 ppm.

### Example 8

### The Preparation of an Anti-Aging Night Cream with Copper Nucleotide and Copper Glycoside

| Water | to 100 |
|---|---|
| Carbomer | 0.2 |
| GMS-SE | 2.0 |
| Stearic Acid | 3.0 |
| Cetyl Alcohol | 1.5 |
| Glycerin | 1.0 |
| Jojoba Oil | 0.1 |
| Sweet Almond Oil | 0.2 |
| Sesame Oil | 0.2 |
| Apricot Kernel Oil | 0.2 |
| Panthenol | 0.0001 |
| Glydant Plus (Preservative) | 0.2 |
| Dimethicone | 2.0 |
| Vitamin E Acetate | 0.0001 |
| Vitaniin A Palmitate | 0.0001 |
| Copper Amino Acid Chelate | 0.025 |
| Adenosine Triphosphate (ATP) | 0.1 |
| Fructose-1,6-diphosphate | 0.1 |
| Glutathione | 0.05 |
| Fragrance | 0.15 |
| Botanical Extract | 0.25 |

Procedure: Copper amino acid chelate and ATP were dissolved in part of water (5% water). Fructose-1,6-diphosphate and glutathione were then added to it and the mixture stirred. It formed a precipitate of copper-ATP-glutathione and copper-fructose diphosphate-glutathione complexes. All other ingredients except fragrance and botanical extract were mixed separately and heated at 70 to 80C, then cooled to room temperature. The trace metal complex pre-blend made above, fragrance, and botanical blends were all added to the main batch and the batch mixed. A light blue cream was obtained with copper content of 30 ppm.

### Example 9

### Copper Glycoside Face & Body Cleanser with Different Donor Sources of Copper

| Water | to 100 |
|---|---|
| Germall II | 0.1 |
| Kathon CG | 0.06 |
| Sodium Lauryl Sulfate | 18.0 |
| Cocamidopropyl betaine | 10.0 |
| Citric Acid | 0.15 |
| Copper Gluconate | 0.025 |
| Copper Amino Acid Chelate | 0.025 |
| Fructose-1,6-diphosphate | 0.2 |
| Fragrance | 0.5 |
| Botanical Extracts | 0.2 |

Procedure: All copper donors were dissolved in part of water (5% water) from the batch. Fructose diphosphate was then added to it with mixing to form the pre-blend. All remaining ingredients were then mixed in a separate tank. The pre-blend was then added to the main batch with mixing. A greenish blue syrupy cleanser product was obtained that contained 65 ppm of Cu.

### Example 10

### Copper Nucleotide and Copper Glycoside Face-Lift Mask with Ascorbic Acid and Lactic Acid as AHA and Zinc as a Cofactor Trace Metal

| PEG-6 | to 100 |
|---|---|
| Aristoflex AVC | 0.8 |
| Deionized Water | 15.0 |
| Copper Gluconate | 0.025 |
| Zinc Gluconate | 0.01 |
| Deionized Water | 1.0 |
| Adenosme Triphosphate (ATP) | 0.2 |
| Glucose monophosphate | 0.2 |
| Ascorbic Acid | 2.0 |
| Silicone Elastomer | 10.0 |
| Chlorophenesin | 0.3 |
| Lactic Acid | 10 |

Procedure: Aristoflex was mixed with deionized water (15% portion) to a clear gel. Copper gluconate, zinc gluconate, ATP, glucose monophosphate, and water (1% portion) were mixed separately to form a light blue pre-blend solution. This was added to the main batch, and all other ingredients were also added to the main batch with mixing. A translucent light blue gel was obtained that had a copper content of 35 ppm and zinc content of 14 ppm.

### Example 11

### Trace Metals Cosmetic Gel (for Antiaging, Anti-Wrinkle, Anti-Acne, Antibacterial, and Anti-Virus Applications)

| Deionized Water | to 100 |
|---|---|
| Xanthan Gum | 1.5 |
| Glutathione | 0.15 |
| Aloe Vera powder | 0.2 |
| Dehydroacetic acid (and) | 0.5 |
| benzyl alcohol | |
| Sodium Hyaluronate | 0.1 |
| Silicone Elastomer | 4.0 |
| Polysorbate-20 | 6.0 |
| Copper Gluconate | 0.23 |
| Zinc Gluconate | 0.23 |
| ATP | 0.55 |
| Deionized Water | 5.0 |
| Glycerine | 40.0 |
| Fragrance | 0.2 |

Procedure: Mix deionized water and xanthan gum till hydrated.

Mix copper gluconate, zinc gluconate, ATP, and deionized water (5.0% portion) to a clear, light blue solution. Add this solution to main batch and mix. Add all other ingredients and mix. A light blue clear gel is obtained with copper content of 322 ppm and zinc content of 322 ppm.

### Example 12

### Trace Metals Clear Serum, High Potency

| Ethoxydiglycol | to 100 |
|---|---|
| Propylene Glycol | 29.8 |
| Deionized Water | 20.0 |
| ATP | 5.51 |
| Copper Gluconate | 2.25 |
| Zinc Gluconate | 1.1 |
| Manganese Gluconate | 1.1 |
| Glutathione | 0.3 |
| Deionized Water | 5.0 |
| Grapefruit extract | 0.1 |
| Fragrance | 0.1 |

Procedure: Mix ATP, Copper gluconate, zinc gluconate, manganese gluconate, and deionized water (20% portion) till a clear greenish blue color is obtained (Premix A). Mix glutathione and deionized water (5.0 portion) in another container till a clear solution is obtained (Premix B). Mix ethoxydiglycol and glycerin in a main batch tank. Add all other ingredients and Premix A and Premix B solutions to main batch tank and mix. Filter this batch to remove any impurities. A greenish blue viscous solution is obtained that has copper content of 3150 ppm, zinc content of 1540 ppm, and manganese content of 1320 ppm. This is used as a high potency serum for eye zone and neck zone applications to remove wrinkles and kill virus.

### Example 13

### Trace Metal Nucleotide Shampoo for Hair Loss Reduction

| Water | to 100 |
|---|---|
| Germall II (preservative) | 0.1 |
| Kathon CG (preservative) | 0.0 |
| Sodium Lauryl Sulfate | 18.0 |
| Cocamidopropyl betaine | 7.0 |
| Citric Acid | 0.1 |
| Copper Gluconate | 0.15 |
| ATP | 0.125 |
| Glutathione | 0.01 |
| Fragrance | 0.5 |

Procedure: All ingredients were mixed together. A clear, light blue viscous liquid was obtained which gave high foam and cleansed hair with less hair loss. It has copper content of 210 ppm.

### Example 14

### Eye Gel with Copper and Zinc Fructose-1,6-diphosphates in an Anhydrous Composition

| | |
|---|---|
| Cyclomethicone | 10.0 |
| Dimethicone | 30.0 |
| Jeesilc 3D5 | 51.8 |
| Tween-20 | 2.0 |
| Glutathione | 0.1 |
| Zinc Gluconate | 0.2 |
| Copper Gluconate | 0.2 |
| Fructose diphosphate | 0.2 |
| PEG-6 | 5.0 |
| Geogard 221 | 0.5 |

Procedure: All ingredients were mixed together till a bluish green suspension product was obtained. The composition needs to be shaken before use for antiaging benefits. It had a copper content of 280 ppm and zinc content of 280 ppm.

I claim:

1. A synergistic cosmetic or pharmaceutical composition for antiaging and antiviral benefits comprising:
(i) A trace metal low molecular weight transporter composition ranging from about 0.0001% to about 10% by weight,
(ii) From about 0.0001% by weight to about 10% by weight of an intracellular storage composition for such trace metal(s),
(iii) From about 0.0001% by weight to about 10.0% by weight of an intracellular energy source for the intracellular transport of such trace metal(s), and,
(iv) From about 1% to about 99% of a cosmetically or pharmaceutically acceptable topical delivery composition.

2. A composition according to claim 1, wherein the trace metal low molecular weight transporter composition is selected from trace metal nucleotides, trace metal phosphorylated saccharides, and trace metal phosphorylated glycosides.

3. A composition according to claim 1, wherein the intracellular trace metal storage composition is selected from a sulfur-containing ingredient, such as glutathione, N-acetyl-cysteine, or a metallothionein.

4. A composition according to claim 1, wherein the trace metal low molecular weight transporter composition and an intracellular energy source for the intracellular transport of trace metal can be same ingredient or composition.

5. A composition according to claim 1, wherein an intracellular energy source for the intracellular transport of trace metal is selected from adenosine triphosphate (ATP), adenosine diphosphate (ADP), adenosine monophosphate (AMP), flavin adenine dinucleotide (FAD), guanosine monophosphate (guanylic acid), guanosine diphosphate, inosine monophosphate (inosinic acid), inosine diphosphate, nicotinamide adenine dinucleotide (NAD), nicotinamide adenine dinucleotide reduced (NADH), citicholine, glucose-1-phosphate, glucose-6-phosphate, glucose-1,6-diphosphate, fructose-1-phosphate, fructose-6-phosphate, fructose-1,6-diphosphate, sucrose phosphate, and combinations thereof.

6. A composition according to claim 1, wherein a cosmetically or pharmaceutically acceptable topical delivery system is selected from a lotion, cream, gel, aerosol, serum, mask, fluid, solution, emulsion, suspension, adsorption mixtures, clay, multi-phase, and multi-component compositions, and anhydrous compositions.

7. A composition according to claim 2, wherein the trace metal low molecular weight transporter composition of a nucleotide, phosphorylated saccharide, or phosphorylated glycoside molecule is made by an in-situ process by the combination of a trace metal acceptor composition with a trace metal donor composition.

8. A composition according to claim 2, wherein the trace metal low molecular weight transporter composition is selected from trace metal adenosine triphosphate (ATP), trace metal adenosine diphosphate (ADP), trace metal adenosine monophosphate (AMP), trace metal flavin adenine dinucleotide (FAD), trace metal guanosine monophosphate (guanylic acid), trace metal guanosine diphosphate, trace metal inosine monophosphate (inosinic acid), trace metal inosine diphosphate, trace metal nicotinamide adenine dinucleotide (NAD), trace metal nicotinamide adenine dinucleotide reduced (NADH), and trace metal citicholine.

9. A composition according to claim 2, wherein the trace metal low molecular weight transporter composition is selected from trace metal glucose-1-phosphate, trace metal glucose-6-phosphate, trace metal glucose-1,6-diphosphate, trace metal fructose-1-phosphate, trace metal fructose-6-phosphate, trace metal fructose-1,6-diphosphate, trace metal sucrose phosphate, and combinations thereof.

10. A composition according to claim 7, wherein the trace metal donor is selected from inorganic or organic derivatives of trace metals or combinations thereof.

11. A composition according to claim 7, wherein the trace metal acceptor composition is selected from a nucleotide, phosphorylated saccharide, phosphorylated glycoside, and combinations thereof.

12. A composition according to claim 8, wherein the trace metal is selected from copper, zinc, manganese, and combinations thereof.

13. A composition according to claim 9, wherein the trace metal is selected from copper, zinc, manganese, and combinations thereof.

14. A composition according to claim 10, wherein the trace metal donor is selected from copper chloride, copper sulfate, copper nitrate, copper acetate, copper glycinate, copper histidinate, copper amino acid chelate, copper peptide, copper gluconate, copper ketoglutarate, copper arginate, copper ascorbate, copper aspartate, copper caprylate, copper citrate, copper cysteinate, copper fumarate, copper glutamate, copper glycerophosphate, copper lactate, copper lysinate, copper malate, copper methionate, copper niacinate, copper picolinate, copper proteinate, copper pyruvate, copper salicylate, copper succinate, copper tartrate, copper yeast complex, and combinations thereof.

15. A composition according to claim 10, wherein the trace metal donor is selected from zinc chloride zinc sulfate, zinc nitrate, zinc acetate, zinc glycinate, zinc histidinate, zinc amino acid chelate, zinc peptide, zinc gluconate, zinc ketoglutarate, zinc arginate, zinc ascorbate, zinc aspartate, zinc caprylate, zinc citrate, zinc cysteinate, zinc fumarate, zinc glutamate, zinc glycerophosphate, zinc lactate, zinc lysinate, zinc malate, zinc methionate, zinc niacinate, zinc picolinate, zinc proteinate, zinc pyruvate, zinc salicylate, zinc succinate, zinc tartrate, zinc yeast complex, and combinations thereof.

16. A composition according to claim 10, wherein the trace metal donor is selected from manganese chloride manganese sulfate, manganese nitrate, manganese acetate, manganese glycinate, manganese histidinate, manganese amino acid chelate, manganese peptide, manganese gluconate, manganese ketoglutarate, manganese arginate, manganese ascorbate, manganese aspartate, manganese caprylate, manganese citrate, manganese cysteinate, manganese fumarate, manganese glutamate, manganese glycerophosphate, manganese lactate, manganese lysinate, manganese malate, manganese methionate, manganese niacinate, manganese picolinate, manganese proteinate, manganese pyruvate, manganese salicylate, manganese succinate, manganese tartrate, manganese yeast complex, and combinations thereof.

17. A composition according to claim 11, wherein the trace metal acceptor composition is selected from adenosine triphosphate (ATP), adenosine diphosphate (ADP), adenosine monophosphate (AMP), flavin adenine dinucleotide (FAD), guanosine monophosphate (guanylic acid), guanosine diphosphate, inosine monophosphate (inosinic acid), inosine diphosphate, nicotinamide adenine dinucleotide (NAD), nicotinamide adenine dinucleotide reduced (NADH), citicholine, glucose-1-phosphate, glucose-6-phosphate, glucose-1,6-diphosphate, fructose-1-phosphate, fructose-6-phosphate, fructose-1,6-diphosphate, sucrose phosphate, and combinations thereof.
